(19) United States
(12) Patent Application Publication
(10) Pub. No.: US 2025/0349486 A1
(43) Pub. Date: Nov. 13, 2025

(54) COMPACT MODE CONVERTER WAVEGUIDE

(71) Applicant: Varian Medical Systems, Inc., Palo Alto, CA (US)

(72) Inventor: Ogy SABEV, San Jose, CA (US)

(73) Assignee: Varian Medical Systems, Inc., Palo Alto, CA (US)

(21) Appl. No.: 18/659,419

(22) Filed: May 9, 2024

(51) Int. Cl. H01J 25/58; H01J 23/20; H01J 23/48

(52) U.S. Cl. CPC H01J 25/58; H01J 23/20; H01J 23/48

(57) ABSTRACT

A radiation support system for radiation therapy includes a waveguide assembly. The waveguide assembly includes a waveguide extending along a first axis, a mode launcher extending along a second axis, the second axis being different than the first axis, and a resonant cavity twist coupling the waveguide to the mode launcher.

### TECHNICAL FIELD

Example embodiments relate to radiation support systems including a magnetron.

### BACKGROUND

In radiosurgery or radiotherapy (collectively referred to as radiation therapy) very intense and precisely collimated doses of radiation are delivered to a target region (volume of tumorous tissue) in the body of a patient in order to treat or destroy tumors or other lesions such as blood clots, cysts, aneurysms or inflammatory masses, for example. The goal of radiation therapy is to accurately deliver a prescribed radiation dose to the tumor/lesion and spare the surrounding healthy tissue. The geometric accuracy of patient positioning relative to the treatment beam, as well as the location and amount of dose delivered to the patient is therefore important. There are a number of factors that could affect geometric and dose delivery accuracy, such as incorrect patient alignment relative to the treatment beam, misalignment of the light field versus radiation field, shift of the skin marker, patient movement, etc.

### SUMMARY

One or more example embodiments relate to a compact mode converter waveguide for a radiation support system.

At least one example embodiment provides an apparatus including a waveguide extending along a first axis, a mode launcher extending along a second axis, the second axis being different than the first axis, and a resonant cavity twist coupling the waveguide to the mode launcher.

The waveguide may be rectangular.

The resonant cavity twist may be a single step resonant cavity twist.

The resonant cavity twist may include a double ridge design.

The waveguide may include a first portion between the mode launcher and the resonant cavity twist and a second portion on an opposite side of the resonant cavity twist from the mode launcher.

The mode launcher may have a cylindrical inner surface.

The cylindrical inner surface of the mode launcher may include a back cavity.

The apparatus may further include an iris between the mode launcher and the waveguide.

A cross section of the iris may have a first area smaller than a second area of a cross section of the waveguide.

The mode launcher may include a top surface that is higher than a top surface of the waveguide.

The apparatus may further include a plurality of pins protruding from at least one of the waveguide, the mode launcher, or the resonant cavity twist.

The mode launcher may include a fin inside the mode launcher running at least partly along a length of the mode launcher.

At least one example embodiment provides a radiation support system including a waveguide assembly, the waveguide assembly including a waveguide extending along a first axis, a mode launcher extending along a second axis, the second axis being different than the first axis, and a resonant cavity twist coupling the waveguide to the mode launcher.

The resonant cavity twist may be a single step resonant cavity twist.

The resonant cavity twist may include a double ridge design.

The waveguide may include a first portion between the mode launcher and the resonant cavity twist and a second portion on an opposite side of the resonant cavity twist from the mode launcher.

The mode launcher may have a cylindrical inner surface.

The cylindrical inner surface of the mode launcher may include a back cavity.

The waveguide assembly may further include an iris between the mode launcher and the waveguide.

A cross section of the iris may have a first area smaller than a second area of a cross section of the waveguide.

### BRIEF DESCRIPTION OF THE DRAWINGS

Other objects and features of the present invention will become apparent from the following detailed description considered in conjunction with the accompanying drawings.

The drawings, however, are only examples and schematic solely for the purpose of illustration and do not limit the present invention. In the drawings:

FIG. 1 illustrates a radiation support system according to example embodiments.

FIG. 2 illustrates a beam generator of the radiation support system.

FIG. 3 illustrates a waveguide of the magnetron.

FIG. 4 illustrates an RF mode pattern in an existing waveguide.

FIG. 5 illustrates a waveguide twist.

FIG. 6 illustrates an RF mode pattern in the waveguide according to example embodiments.

FIG. 7 illustrates an RF mode pattern in a typical single step resonant cavity twist according to example embodiments.

FIG. 8 illustrates a cross section of a single step resonant cavity waveguide inside the twist according to example embodiments.

FIG. 9 illustrates an RF mode pattern in a typical orthogonal mode launcher according to example embodiments.

FIG. 10 illustrates a beam generator including a waveguide with the compact mode converter installed according to example embodiments.

FIG. 11 illustrates the compact mode converter waveguide according to example embodiments.

FIG. 12A illustrates a mode launcher according to example embodiments.

FIG. 12B illustrates electric field lines in a mode launcher according to example embodiments.

### DETAILED DESCRIPTION

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

Detailed illustrative embodiments are disclosed herein. However, specific structural and functional details disclosed herein are merely representative for purposes of describing example embodiments. The example embodiments may, however, be embodied in many alternate forms and should not be construed as limited to only the embodiments set forth herein.

When the words “about” and “substantially” are used in this application in connection with a numerical value, it is intended that the associated numerical value include a tolerance of +10% around the stated numerical value, unless otherwise explicitly defined. Further, regardless of whether numerical values are modified as “about” or “substantially,” it will be understood that these values should be construed as including a of +10% around the stated numerical value.

Independent of the grammatical term usage, individuals with male, female or other gender identities are included within the term.

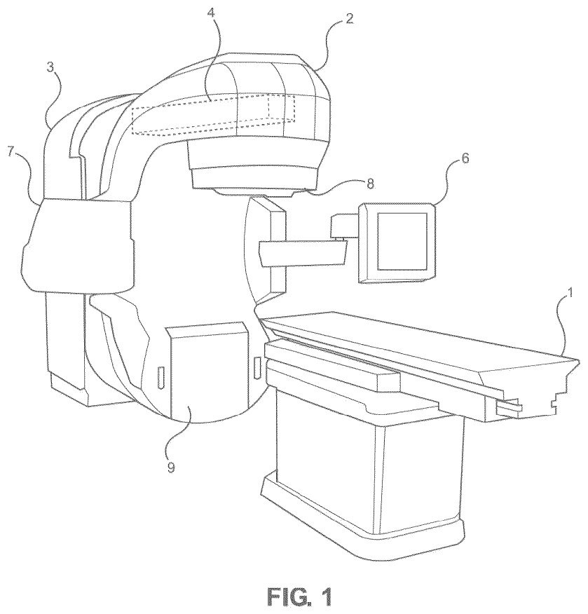

FIG. 1 illustrates a radiation support system according to example embodiments.

Referring to FIG. 1 a radiation support system 10 includes a patient support (e.g., patient couch) 1 that can move the patient, a gantry 2 that circles about one end of the patient couch, a stand 3 that supports the gantry, a beam generator 4 mounted in the gantry, a first image detector 6 mounted to the gantry, and a first imaging radiation source 7 mounted to the gantry opposite to first image detector 6. The radiation support system 10 may be, for example, a Halcyon (HAL) system.

Beam generator 4 generates and accelerates electrons into a beam of electrons. When a tungsten target is used, the electron beam strikes the target and generates X-rays, which are conveyed to the patient treatment area. When the tungsten target is not used, the electron beam is conveyed to the patient treatment area.

The electrons from the beam generator 4 are spatially filtered by an adjustable multi-leaf collimator (MLC) 8 having a plurality of moveable leaves of radiation-absorbent material (e.g., 120 leaves). A treatment beam of electrons or X-Rays emerges from MLC 8, and the beam can have a wide range of cross-sectional patterns, as set by the positions of the leaves of MLC 8. Prior to reaching MLC 8, the treatment beam is passed through a set of jaws, which open and close around the beam. When the jaws are closed, the treatment beam does not emerge from the collimator structure. When the jaws are open, the treatment beam emerges and strikes the patient. The first imaging detector and imaging radiation source lie in a plane that is perpendicular to the treatment beam. The treatment system may also comprise a second imaging detector 9 disposed about 6 feet away and opposite to the treatment beam (in FIG. 1, this second imaging detector is shown in a folded position, folded into a compartment of the gantry). The second imaging detector uses radiation from the treatment beam to provide an image that is perpendicular to that provided by the first imaging detector.

Patient support 1 need not be moveable, and may be a fixed support. Gantry 2 and stand 3 implement one particular form of a beam-positioning mechanism that is capable of holding and/or moving the radiation beam path (e.g., trajectory) with respect to the patient. Other beam-positioning mechanisms are known to the art, and may be used in conjunction with the invention. Beam-positioning mechanisms include, but are not limited to: gantries, ring gantries, robotic arms, beam-steering devices (including those that use electric fields and/or magnetic fields), and combinations thereof. Multi-leaf collimator 8 implements one particular form of a beam-shaping mechanism that is capable of modifying the cross-sectional shape of the radiation beam. Beam-shaping devices include, but are not limited to, multi-leaf collimators, iris collimators, jaw collimators, electric-field shapers (e.g., “electrostatic” shapers), magnetic-field shapers (e.g., “magnetic” lenses), and combinations thereof.

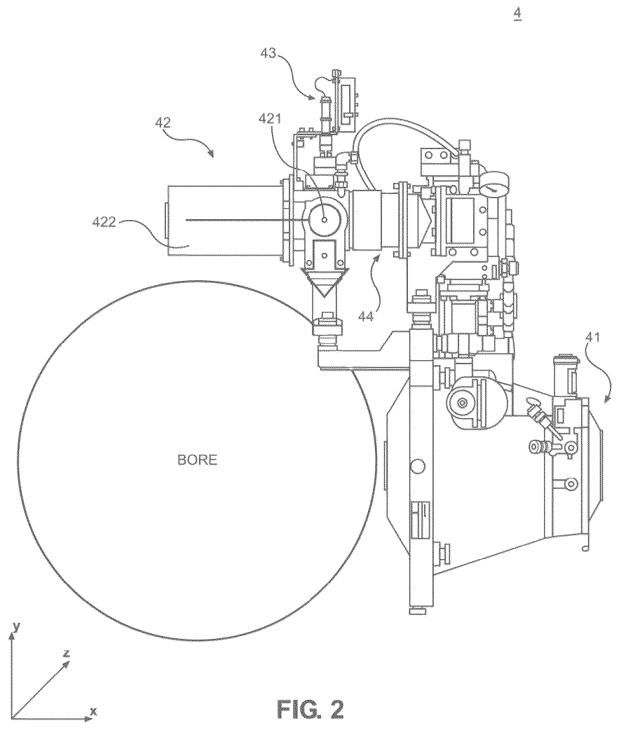

FIG. 2 illustrates a beam generator of the radiation support system.

Beam generator 4 comprises a linear accelerator 41 that generates and accelerates electrons into a beam of electrons and a magnetron or klystron 42 that generates microwave pulse signals for the electrodes of the accelerating structure, for example an E2V magnetron. The magnetron 42 includes a tuning mechanism 43 and a waveguide 44.

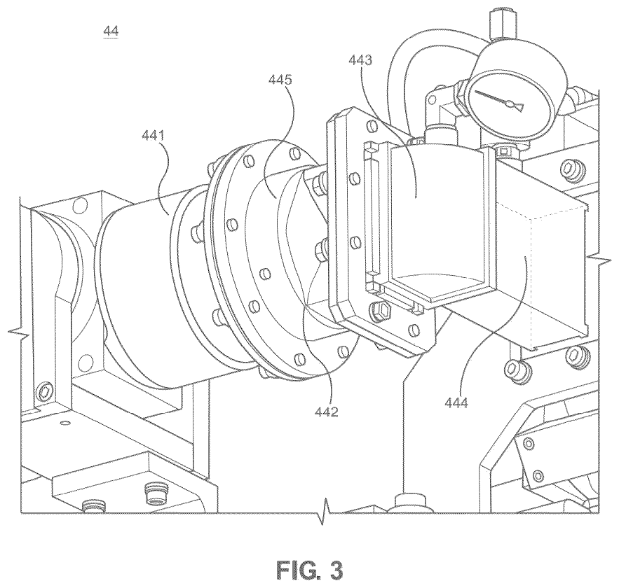

FIG. 3 illustrates a waveguide of the magnetron.

Referring to FIG. 3, the waveguide 44 includes a cylindrical waveguide 441 that converts to a rectangular waveguide 442 via adapter 445, an E-bend 443, and a rectangular waveguide 444.

A significant amount of magnetron failures (specifically regarding machine dose output verses rotation) may be caused by the magnetron 42 (e.g., an E2V magnetron) being oriented 90 degrees from a recommended (or given) position around its own central axis. E2V recommends a central axis of a cylindrical cathode 421 to be oriented orthogonal to an axis of rotation gantry rotation (e.g., direction z) for better structural rigidity. The cathode support 422 is such that at 270 degrees (as defined by the Halcyon machine gantry angle) the lever arm effect of the cathode support 422 results in droop with the current orientation. If rotated there would be less magnetron moding (magnetron oscillating with an improper RF frequency pattern briefly) and dropped pulses due to the inherent way the magnetron operates. There is less droop (mechanical deflection due to gravity) on the cathode due to the mechanical orientation. However, it may be difficult to rotate (e.g., orient) the magnetron 42 based on a form factor of the radiation support system 10. For example, FIG. 2 illustrates an orientation of the magnetron 42 with a central axis of the cylindrical cathode 421 in the direction z, parallel to the axis of gantry rotation in the direction z (e.g., a non-recommended position).

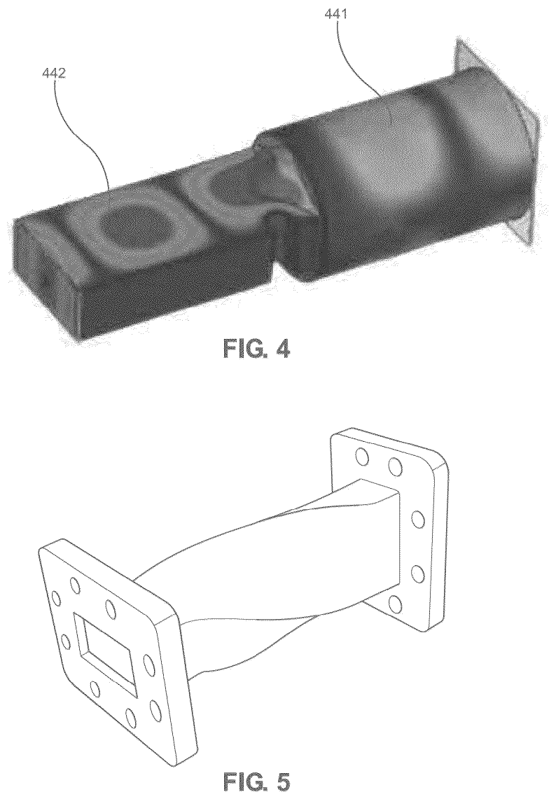

For example, reorienting the magnetron 42 about its central axis by 90 degrees would result in an RF mode pattern output from an antenna structure (not pictured) of the magnetron 42 not lining up with the rectangular waveguide 442 mode pattern orientation. An RF mode pattern of the waveguide 44 is shown in FIG. 4.

Therefore, to orient the magnetron 42 according to the recommended position, the whole waveguide 44 would need to be flipped vertically by 90 degrees (allowing both mode patterns to be in the same direction). However, this may be difficult or impossible due to a form factor of the radiation support system 10 (e.g., a HAL form factor).

A possible alternative solution to rotating the waveguide 44 is a waveguide twist, shown in FIG. 5. However such a device would take too much space for the form factor of the radiation support system 10. For example, the waveguide twist shown in FIG. 5 may have a length of about 6.6 inches. The magnetron cannot be moved up or down in relation to other components of the radiation support system 10 to incorporate these long waveguide twist sections.

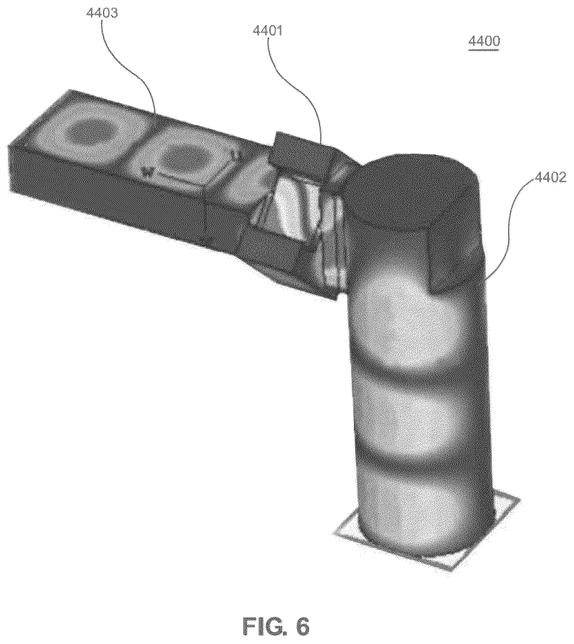

FIG. 6 illustrates a waveguide according to example embodiments.

Referring to FIG. 6, a waveguide 4400 (e.g., a waveguide assembly) includes a rectangular waveguide 4403, a single step resonant cavity twist 4401, and a mode launcher 4402. Waveguide 4400 may be referred to as a compact mode converter waveguide.

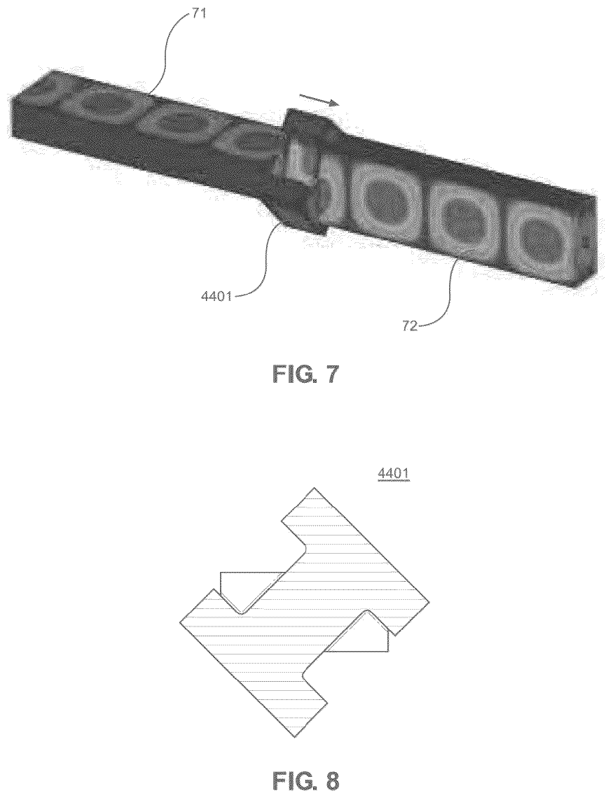

FIG. 7 illustrates a single step resonant cavity twist according to example embodiments.

As shown in FIG. 7, a single step resonant cavity twist 4401 according to example embodiments is shown between two rectangular waveguide sections 71 and 72. The single step resonant cavity twist 4401 may rotate the RF mode pattern in rectangular waveguide section 71 by 90 degrees to the RF mode pattern shown in rectangular waveguide section 72. The RF mode pattern shown in FIG. 7 represents a fundamental TE10 mode of operation through the single step resonant cavity twist 4401.

The single step resonant cavity twist 4401 may have an H-shaped cross section, as shown in FIG. 8. For example, the single step resonant cavity twist 4401 may be a double ridged section of rectangular waveguide at an angle to the main waveguide and may be confined in a pill box resonant structure.

Returning to FIG. 7, the single step resonant cavity twist 4401 may be positioned at an angle oblique to the rectangular waveguide.

The single step resonant cavity twist 4401 is more compact than a waveguide twist (e.g., shown in FIG. 5). For example, the single step resonant cavity twist 4401 may have a length of about 1.6 inches, compared to the length of about 6.6 inches of the waveguide twist shown in FIG. 5. However, the compactness results in a loss of bandwidth.

However there is still limited space to insert such a twist in the existing waveguide 44. For example, there is not much room for the mounting flanges and the single step resonant cavity twist 4401 (which would need an H-bend instead of the E-bend 443). Accordingly, the single step resonant cavity twist 4401 may be paired with the mode launcher 4402.

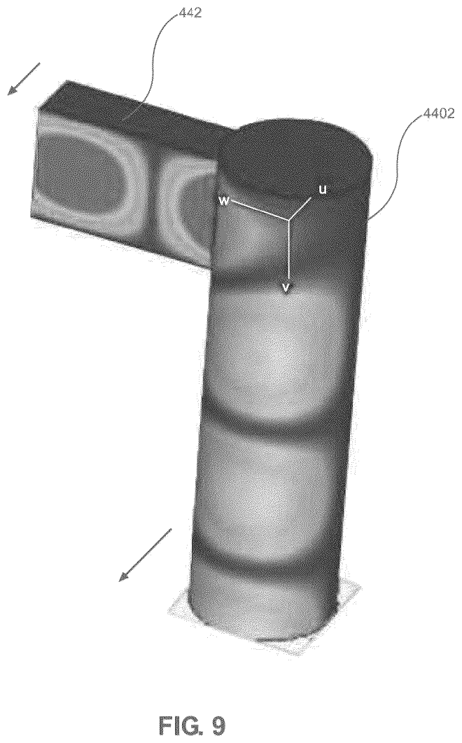

FIG. 9 illustrates a mode launcher according to example embodiments.

Referring to FIG. 9, the mode launcher 4402 launches the RF wave from TE10 mode in the rectangular waveguide 442 into TE11 mode in the circular portion. The mode launcher 4402 is an orthogonal equivalent of the inline cylindrical waveguide 441 currently used to launch from circular to rectangular in the magnetron 42, as shown in FIG. 4. For example, a longitudinal axis of the mode launcher 4402 may be different from a longitudinal axis of the rectangular waveguide 442. For example, the longitudinal axis of the mode launcher 4402 may be orthogonal to the longitudinal axis of the rectangular waveguide 442. The longitudinal axis of the rectangular waveguide 442 may be referred to as a first axis and the longitudinal axis of the mode launcher 4402 may be referred to as a second axis. Mode launchers such as the mode launcher 4402 must have fields in the same direction in the rectangular and circular sections as indicated by the arrows shown in FIG. 9.

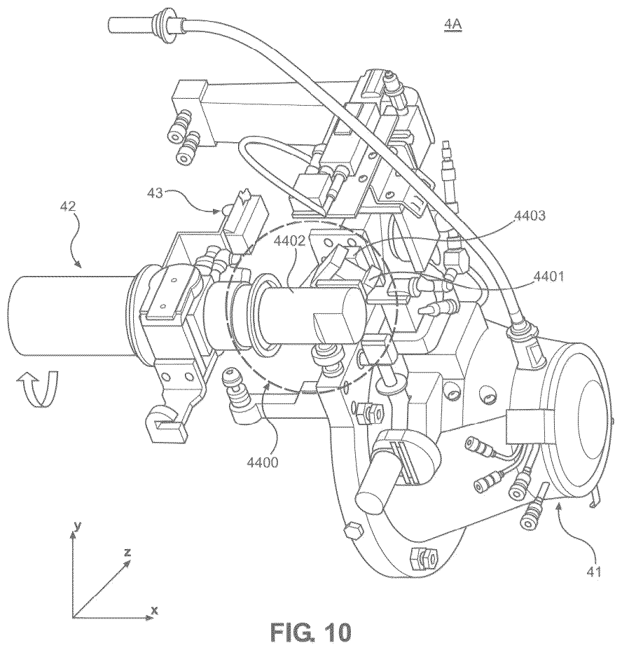

FIG. 10 illustrates a beam generator including a waveguide according to example embodiments.

Referring to FIG. 10, a beam generator 4A according to example embodiments includes linear accelerator 41 and magnetron 42 including tuning mechanism 43 and waveguide 4400.

As shown in FIG. 10, combining the single step resonant cavity twist 4401 with the mode launcher 4402 allows reorientation of the magnetron 42 by 90 degrees around its central axis while still satisfying the necessary RF field pattern in the waveguide network (matching the field pattern emitted from the magnetron) within the form factor of the radiation support system 10.

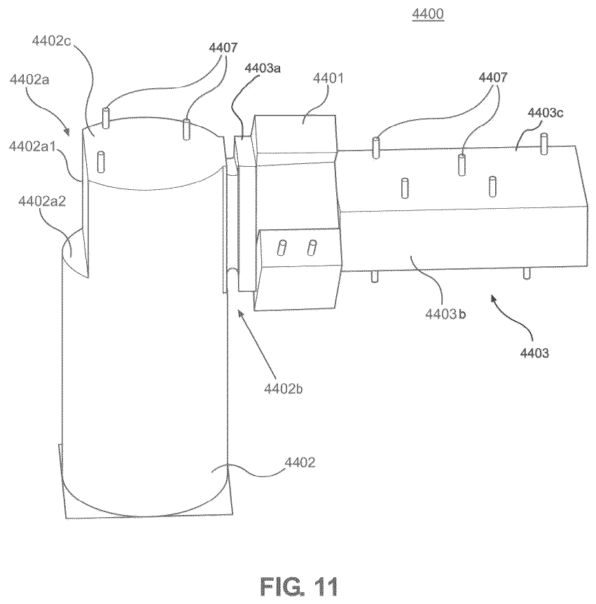

For example, the waveguide 4400 may replace the existing rectangular waveguide 442, E-bend 443, rectangular waveguide 444, and adapter 445 allowing the magnetron 42 to be reoriented by 90 degrees (e.g., tuning mechanism 43 is rotated 90 degrees compared with beam generator 4 shown in FIG. 2) while maintaining the correct RF mode and fitting in the same physical location as the previous rectangular waveguide 442, E-bend 443, rectangular waveguide 444, and adapter 445. As shown in FIG. 11, the single step resonant cavity twist 4401 allows for the ability to rotate the RF field pattern of the mode launcher 4402 to the rectangular waveguide 4403 as shown in FIG. 6.

FIG. 11 illustrates a waveguide according to example embodiments.

FIG. 11 illustrates an inside SF6 volume of a waveguide structure according to example embodiments. For example, FIG. 11 illustrates inner surfaces as if external surfaces of the waveguide 4400 are invisible, and we only see the volume inside.

The mode launcher 4402 may cause some mode spikes of the TE11 RF mode signal if excited in the wrong orientation. The waveguide 4400 may be modified to include various features for suppressing these undesirable mode spikes.

For example, referring to FIG. 11, the waveguide 4400 may include a back cavity 4402a, an iris 4402b, and/or pins 4407.

The mode launcher 4402 may be a generally cylindrical shape except for the back cavity 4402a. For example, an inside surface of the mode launcher 4402 may be a generally cylindrical shape. The back cavity 4402a may be arranged at an upper portion of the mode launcher 4402 at a similar height to the single step resonant cavity twist 4401 and the rectangular waveguide 4403. The back cavity 4402a may be a cutout portion of a curved surface of the cylindrical shape of the mode launcher 4402. For example, the back cavity 4402a may include a first flat portion 4402a1 of the inner surface of the mode launcher 4402 opposite from the single step resonant cavity twist 4401 and the rectangular waveguide 4403. The first flat portion 4402a1 may extend downward longitudinally to a second flat portion 4402a2, orthogonal to the first flat portion 4402a1.

The back cavity 4402a may change the impedance of the waveguide 4400 allowing it to match the impedance of the rectangular aperture into the single step resonant cavity twist 4401. It thus allows the RF wave to flow from a cylinder section to a rectangular section without causing a reflection in the process.

The back cavity 4402a may therefore increase bandwidth and push undesirable modes up in frequency. The shape of the back cavity 4402a reinforces the existing electric field patterns of the desired mode and is orthogonal to the field pattens of the undesirable mode to be suppressed.

The iris 4402b may be between the mode launcher 4402 and the rectangular waveguide 4403. The rectangular waveguide 4403 may include a first portion 4403a between the iris 4402b and the single step resonant cavity twist 4401 and a second portion 4403b on an opposite side of the single step resonant cavity twist 4401 from the first portion 4403a. The mode launcher 4402 may be connected to the first portion 4403a via the iris 4402b.

The iris 4402b is a rectangular portion of the waveguide 4400. An area of a cross section of the iris 4402b is smaller than an area of a cross section the rectangular waveguide 4403. The iris 4402b may modify the waveguide impedance at the waveguide junction. The iris 4402b may be about 0.05″ to about 0.3″ thick and may protrude about 0.05″ to about 0.5″ or more inches into the waveguide 4400. The aspect ratio may deviate from rectangular. For example, one pair of the iris walls may protrude more or less. For example the resulting aperture may be square.

The mode launcher 4402 includes a top surface 4402c. The top surface 4402c may be circular. Alternatively, the top surface 4402c may be mostly circular with a flattened portion corresponding with the back cavity 4402a. As shown in FIG. 11, the top surface 4402c of the mode launcher 4402 may be higher than a top surface 4403c of the rectangular waveguide 4403. For example, the top surface 4402c may be similar to a tuning stub section. The top surface 4402c may be about 0.05″ to about 1″, or more, higher than the top surface 4403c of the rectangular waveguide 4403.

The reflected waves from the stub section may interfere constructively with the electric fields of the desired mode, but may not interfere destructively and may not support the field patterns of the undesirable mode spikes.

The waveguide 4400 may include pins 4407 protruding partially or fully from the top and/or the bottom of the waveguide 4400 in various places. For example, the pins 4407 may protrude from components of the waveguide such as the rectangular waveguide 4403, the mode launcher 4402 (e.g., a top surface of the mode launcher 4402), and/or the single step resonant cavity twist 4401. FIG. 11 illustrates a plurality of pins 4407, however example embodiments are not limited to the number or placements of the pins 4407 shown in FIG. 11. For example, the waveguide 4400 may include more or fewer pins 4407. The pins 4407 may be made of a metal (e.g., a same metal as the waveguide 4400) and may be generally cylindrically shaped.

The pins 4407 may protrude from a top surface of a component of the waveguide 4400 and bottom surface of the component, connecting (e.g., electrically connecting) the top surface to the bottom surface. This connection may provide a path for electric current to follow, bypassing part or all of the wall current changing the impedance. The pins 4407 may be bolted and/or brazed to the top and/or the bottom of the components of the waveguide 4400.

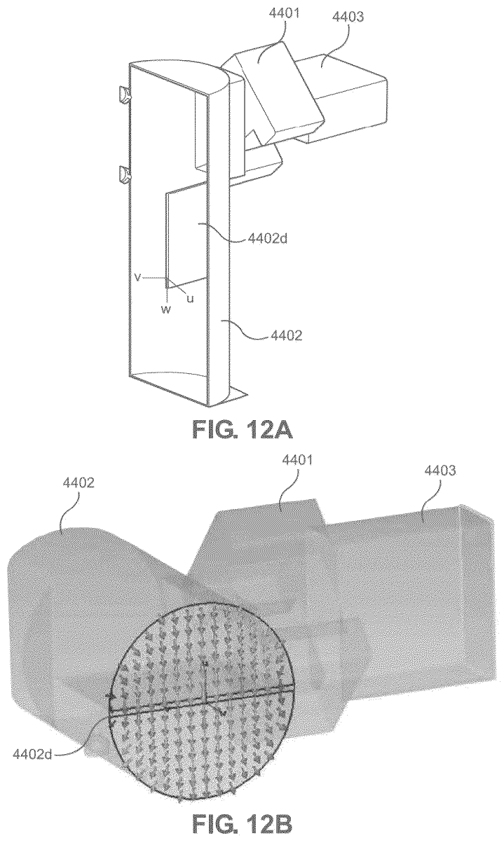

FIG. 12A illustrates a mode launcher according to example embodiments.

Referring to FIG. 12A, the mode launcher 4402 may include a fin 4402d. The fin 4402d may be inside the mode launcher 4402 running along the length of the mode launcher 4402. The example shown in FIG. 12A illustrates the fin 4402d running along a portion of the length of the mode launcher 4402. However, example embodiments are not limited thereto and the fin 4402d may be longer or shorter. For example, the fin 4402d may run along less than half the length of the mode launcher 4402, or the fin 4402d may run along more than half the length of the mode launcher 4402 (e.g., the full length of the mode launcher 4402). The fin 4402d may be relatively thin. For example, the fin 4402d may be about 0.100 inches thick, or less. The fin 4402d may be comprised of any metal with good conductivity (e.g., copper).

FIG. 12B illustrates electric field lines in a mode launcher according to example embodiments.

Because of the axisymmetric nature of a waveguide (e.g., the mode launcher 4402), RF waves of a mode (e.g., a TE11 mode) can be unstable and prone to polarization. As shown in FIG. 12B, the fin 4402d may orthogonally bifurcate the electric field lines of the TE11 mode RF waves, indicated by arrows in FIG. 12B, inside the mode launcher 4402. The fin 4402d may therefore act as a mode suppressor to stabilize the TE11 mode RF waves inside the mode launcher 4402.

Although the present invention has been described in detail with reference to example embodiments, the present invention is not limited by the disclosed examples from which the skilled person is able to derive other variations without departing from the scope of the invention.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Specific structural and functional details disclosed herein are merely representative for purposes of describing example embodiments. This invention may, however, be embodied in many alternate forms and should not be construed as limited to only the embodiments set forth herein.

### Non-Limiting Illustrative Embodiments

The following is a list of non-limiting illustrative embodiments disclosed herein:

Illustrative embodiment 1. An apparatus including a waveguide extending along a first axis, a mode launcher extending along a second axis, the second axis being different than the first axis, and a resonant cavity twist coupling the waveguide to the mode launcher.

Illustrative embodiment 2. The apparatus of illustrative embodiment 1, wherein the waveguide is rectangular.

Illustrative embodiment 3. The apparatus of any of the preceding embodiments, wherein the resonant cavity twist is a single step resonant cavity twist.

Illustrative embodiment 4. The apparatus of any of the preceding embodiments, wherein the resonant cavity twist includes a double ridge design.

Illustrative embodiment 5. The apparatus of any of the preceding embodiments, wherein the waveguide includes a first portion between the mode launcher and the resonant cavity twist and a second portion on an opposite side of the resonant cavity twist from the mode launcher.

Illustrative embodiment 6. The apparatus of any of the preceding embodiments, wherein the mode launcher has a cylindrical inner surface.

Illustrative embodiment 7. The apparatus of illustrative embodiment 6, wherein the cylindrical inner surface of the mode launcher includes a back cavity.

Illustrative embodiment 8. The apparatus of any of the preceding embodiments, further including an iris between the mode launcher and the waveguide.

Illustrative embodiment 9. The apparatus of illustrative embodiment 8, wherein a cross section of the iris has a first area smaller than a second area of a cross section of the waveguide.

Illustrative embodiment 10. The apparatus of any of the preceding embodiments, wherein the mode launcher includes a top surface that is higher than a top surface of the waveguide.

Illustrative embodiment 11. The apparatus of any of the preceding embodiments, further including a plurality of pins protruding from at least one of the waveguide, the mode launcher, or the resonant cavity twist.

Illustrative embodiment 12. The apparatus of any of the preceding embodiments, wherein the mode launcher includes a fin inside the mode launcher running at least partly along a length of the mode launcher.

Illustrative embodiment 13. A radiation support system including a waveguide assembly, the waveguide assembly including a waveguide extending along a first axis, a mode launcher extending along a second axis, the second axis being different than the first axis, and a resonant cavity twist coupling the waveguide to the mode launcher.

Illustrative embodiment 14. The radiation support system of illustrative embodiment 13, wherein the resonant cavity twist is a single step resonant cavity twist.

Illustrative embodiment 15. The radiation support system of either of illustrative embodiment 13 or illustrative embodiment 14, wherein the resonant cavity twist includes a double ridge design.

Illustrative embodiment 16. The radiation support system of any of illustrative embodiments 13-15, wherein the waveguide includes a first portion between the mode launcher and the resonant cavity twist and a second portion on an opposite side of the resonant cavity twist from the mode launcher.

Illustrative embodiment 17. The radiation support system of any of illustrative embodiments 13-16, wherein the mode launcher has a cylindrical inner surface.

Illustrative embodiment 18. The radiation support system of illustrative embodiment 17, wherein the cylindrical inner surface of the mode launcher includes a back cavity.

Illustrative embodiment 19. The radiation support system of any of illustrative embodiments 13-18, wherein the waveguide assembly further includes an iris between the mode launcher and the waveguide.

Illustrative embodiment 20. The radiation support system of illustrative embodiment 19, wherein a cross section of the iris has a first area smaller than a second area of a cross section of the waveguide.

What is claimed is:

1. An apparatus comprising:
a waveguide extending along a first axis;
a mode launcher extending along a second axis, the second axis being different than the first axis; and
a resonant cavity twist coupling the waveguide to the mode launcher.

2. The apparatus of claim 1, wherein the waveguide is rectangular.

3. The apparatus of claim 1, wherein the resonant cavity twist is a single step resonant cavity twist.

4. The apparatus of claim 1, wherein the resonant cavity twist includes a double ridge design.

5. The apparatus of claim 1, wherein the waveguide includes a first portion between the mode launcher and the resonant cavity twist and a second portion on an opposite side of the resonant cavity twist from the mode launcher.

6. The apparatus of claim 1, wherein the mode launcher has a cylindrical inner surface.

7. The apparatus of claim 6, wherein the cylindrical inner surface of the mode launcher includes a back cavity.

8. The apparatus of claim 1, further comprising:
an iris between the mode launcher and the waveguide.

9. The apparatus of claim 8, wherein a cross section of the iris has a first area smaller than a second area of a cross section of the waveguide.

10. The apparatus of claim 1, wherein the mode launcher includes a top surface that is higher than a top surface of the waveguide.

11. The apparatus of claim 1, further comprising a plurality of pins protruding from at least one of the waveguide, the mode launcher, or the resonant cavity twist.

12. The apparatus of claim 1, wherein the mode launcher includes a fin inside the mode launcher running at least partly along a length of the mode launcher.

13. A radiation support system comprising:
a waveguide assembly including,
a waveguide extending along a first axis,
a mode launcher extending along a second axis, the second axis being different than the first axis, and
a resonant cavity twist coupling the waveguide to the mode launcher.

14. The radiation support system of claim 13, wherein the resonant cavity twist is a single step resonant cavity twist.

15. The radiation support system of claim 13, wherein the resonant cavity twist includes a double ridge design.

16. The radiation support system of claim 13, wherein the waveguide includes a first portion between the mode launcher and the resonant cavity twist and a second portion on an opposite side of the resonant cavity twist from the mode launcher.

17. The radiation support system of claim 13, wherein the mode launcher has a cylindrical inner surface.

18. The radiation support system of claim 17, wherein the cylindrical inner surface of the mode launcher includes a back cavity.

19. The radiation support system of claim 13, wherein the waveguide assembly further includes an iris between the mode launcher and the waveguide.

20. The radiation support system of claim 19, wherein a cross section of the iris has a first area smaller than a second area of a cross section of the waveguide.
